(19) United States
(12) Patent Application Publication
(10) Pub. No.: US 2021/0018896 A1
(43) Pub. Date: Jan. 21, 2021

(54) Methods and Systems for Noninvasive Mind-Controlled Devices

(71) Applicant: CARNEGIE MELLON UNIVERSITY, Pittsburgh, PA (US)

(72) Inventors: Bin He, Pittsburgh, PA (US); Brad J. Edelman, Pittsburgh, PA (US); Jianjun Meng, Pittsburgh, PA (US); Daniel Suma, Pittsburgh, PA (US)

(73) Assignee: CARNEGIE MELLON UNIVERSITY, Pittsburgh, PA (US)

(21) Appl. No.: 16/931,408

(22) Filed: Jul. 16, 2020

Related U.S. Application Data
(60) Provisional application No. 62/921,963, filed on Jul. 16, 2019.

(51) Int. Cl. G05B 19/409; A61B 5/0476; A61B 5/04; A61B 5/00; G09B 19/00

(52) U.S. Cl. CPC G05B 19/409; A61B 5/0476; A61B 5/04008; G05B 2219/36133; A61B 5/7264; G09B 19/00; A61B 5/04012

(57) ABSTRACT

A system and method comprising a noninvasive framework utilizing electroencephalography (EEG) to achieve the neural control of a robotic device for continuous random target tracking.

### CROSS-REFERENCE TO RELATED APPLICATIONS

This application claims the benefit under 35 U.S.C. § 119 of Provisional Application Ser. No. 62/921,963, filed Jul. 16, 2019, which is incorporated herein by reference.

### STATEMENT REGARDING FEDERALLY SPONSORED RESEARCH

This invention was made with government support under 7R01AT009263 awarded by the National Institutes of Health. The government has certain rights in the invention.

### BACKGROUND OF THE INVENTION

The invention relates generally to a system and method to control an external device through a noninvasive brain-computer interface (BCI). Mind-controlled assistive devices such as robots are of practical use for patients who are paralyzed or with motor dysfunctions, and even for the general population.

Brain-computer interfaces (BCIs) utilizing signals acquired with intracortical implants have achieved successful high-dimensional robotic device control useful for completing daily tasks. However, the substantial amount of medical and surgical expertise required to correctly implant and operate these systems significantly limits their use beyond a few clinical cases. A noninvasive counterpart requiring less intervention that can provide high-quality control would profoundly impact the integration of BCIs into the clinical and home setting. Noninvasive BCI technology detects a human's mental intent or state (“mind”) by recording brain signals noninvasively and decodes this “mind” to translate thoughts into the control of external devices for various purposes. Such “mind-controlled” devices open the door to improving the lives of patients suffering from various neurological disorders, including amyotrophic lateral sclerosis and spinal cord injury, as well as those suffering from stroke. This technology may also be used for therapeutic or rehabilitative applications, and even for educational and entertainment games. In all, BCI offers a direct communication channel between a brain and external devices, bypassing the neuromuscular system.

### BRIEF SUMMARY

According to embodiments of the present invention is a noninvasive framework utilizing electroencephalography (EEG) to achieve the neural control of a robotic device for continuous random target tracking. This framework addresses and improves upon both the “brain” and “computer” components by respectively increasing user engagement through a continuous pursuit task and associated training paradigm, and the spatial resolution of noninvasive neural data through EEG source imaging. In all, the framework enhanced BCI learning by nearly 60% for traditional center-out tasks and by over 500% in the more realistic continuous pursuit task. We further demonstrated an additional enhancement in BCI control of almost 10% by using online noninvasive neuroimaging. Finally, this framework was deployed in a physical task, demonstrating a near seamless transition from the control of an unconstrained virtual cursor to the real-time control of a robotic arm. Such combined advances in the quality of neural decoding and the practical utility of noninvasive robotic arm control will have major implications on the eventual development and implementation of neurorobotics by means of noninvasive BCI.

This invention presents novel methodologies for controlling external devices by means of noninvasive approaches. Electrophysiological signals including electroencephalography (EEG) and magnetoencephalography (MEG) are used to record and decode human's mental intent or state, through a variety of techniques based on the spatio-temporal-spectral signatures contained within the EEG/MEG signals to reveal the brain state or mental intent of a human subject. Such processed signals are then fed into external devices to control “actions” of said devices, such as the continuous or discretized movement of a robotic device, or other complex functional or movement-based tasks.

This technology represents a hybrid framework integrating multiple approaches to optimize the performance of noninvasive BCI, including imagery paradigms, spatio-temporal-spectral decoding schemes to extract brain signals representing a subject's intention, and a continuous pursuit task and training paradigm. It uses real-time source imaging to enhance signal quality in the context of detecting and decoding “intention and state” related signals. This framework enables the accomplishment of external device control by means of a human's “mind” that exceeds the performance of other noninvasive BCI.

In one embodiment, brain “intent” signals are detected using a plurality of sensors that record the electrical signals, magnetic signals or even hemodynamic signals produced by the neural activations associated with “intent”. The sensors may be electrodes for electrical recordings, or magnetic field detectors for magnetic recordings. The neural “sources” that are responsible for the scalp electrical/magnetic signals are estimated through a real-time source imaging approach. The waveforms of source signals in related brain regions of interest that are associated with the imagery tasks are extracted, processed in the temporal, spatial and spectral domains, and used to control an external device. For electrical signals, the relationship between scalp electroencephalography (EEG) and brain sources is solved through EEG source imaging, where a forward head model is used and governed by Poisson's equation with regard to electric potential. For magnetic signals, the relationship between scalp magnetoencephalography (MEG) and brain sources is solved through MEG source imaging, where a forward head model is used and governed by Poisson's equation with regard to magnetic potential or field. The magnetic recordings may be collected by use of portable MEG probes placed outside of the scalp, such as optically-pumped magnetometers. The brain sources are estimated by source imaging from MEG signals.

In another embodiment, a plurality of source signals are used to compute signals reflecting the “intent” of human subjects, after further processed in the time, frequency, or spatial domains, and include extracting the event related synchronization (ERS) or event related desynchronization (ERD) signals. In another embodiment, brain intent signals are detected using a plurality of ear EEG electrodes, where electrodes are placed on or in the vicinity of the ears. With temporal-spectral processing, brain “intent and state” signals are extracted from ear EEG recordings to control external devices. In another embodiment, brain intent signals are detected using a plurality of electrodes placed over the forehead to extract brain “intention” signals, and processed signals used to decode “intention” or state. In another embodiment, subjects are trained with continuous pursuit paradigms to enhance subject engagement during the training to enhance the performance and speed up the BCI skill acquisition. In another embodiment, spatio-temporal-spectral features are decoded by linear or nonlinear static/adaptive classifiers. The linear classifier can include simple linear combination of powers, linear discriminative analysis, support vector machine with linear kernels and etc. The nonlinear classifier can include neural networks or deep learning networks, support vector machine with nonlinear kernels, etc. The adaptive technique can include adaptation based on simple assumption of zero mean and unit variance, supervised adaptation based on historical recording data and labels, semi-supervised adaptation based on the combination of training data with labels and online testing data with estimated labels, unsupervised adaptation based on testing data with estimated labels, etc.

This technology addresses the challenge of noninvasive BCI for continuously controlling a robotic device. It improves the signal-to-noise ratio of noninvasive EEG signals using a hybrid source imaging and spectral filter to decode and extract “intention” signals that are virtually mapped to brain regions responsible for generating “imagery” tasks. The continuous control paradigm increases and maintains user engagement, a cognitive component known to affect task performance, throughout device control. The technology has been demonstrated using motor imagery tasks but is applicable to other forms of cognitive tasks, such as the imagery of “images”, computational tasks, abstract thoughts, etc., or a combination of multiple tasks. The technology also offers additional efficiency and speed of robotic arm/device task completion by using the continuous paradigm. One of the drawbacks of previous demonstrations is that they mostly require discrete trial paradigms, which, when used in practical situations, expand sequentially downstream and quick mental tasks into extended sequences that take longer to complete and are less flexible for correcting mistakes.

One example application of this technology is to develop “mind-controlled” assistive robotic devices, where a human subject's intention is recorded using EEG and decoded using the present technology to extract reliable control signals, and control the actions of assistive robotic devices. Such assistive robotic devices include a robotic arm for reaching, grasping an object, and continuously moving, and performing actions under control of a human subject's intention. This technology can also be used to control a rehabilitative device to help disabled or paralyzed subjects to rehabilitate her/his motor functions or regain its motor functions. Another application of this technology is to develop “mind-controlled” smart devices that can be controlled by signals from a human subject's brain, using “imagery and state” tasks (including motor imagery or other cognitive tasks).

Another example application of this technology is to develop “mind-controlled” neuroprosthetic limbs to control a prosthetic limb of a subject using the intention signals extracted from the subject. Another application of this technology includes controlling functions of a car during driving without using the hands of human driver, alerting a human driver based on the brain status as decoded from EEG signals, controlling an electronic device in an office setting, a house-setting, or industrial setting using the “intention” signals. Another application of this technology includes controlling a wheelchair by a patient for movement without the active involvement of limbs. Another application of this technology includes controlling a smart phone using the “intention” of a human subject.

Another application of this technology is to use it for brain training, so it could reduce cognitive declines or help recovery from mental disorders. Another application of this technology is to use it to provide neural feedback and adjust educational practices which account for a user's specific mental state. Another application of this technology includes controlling communication devices to convey the human “intention” to other parties without speaking or writing using hands. Another application of this technology includes controlling a drone or moving object using the “intention” signals decoded from a human subject.

Another application of this technology includes mind-controlled video games (or other educational and entertainment software) with mind only, or using both mind and hands to play games using the “intention” signals decoded from a human subject. It includes such device in regular display or in virtual reality or augmented reality setups. Another application of this technology includes using BCI for accessing effectiveness or progression of education and training. Another application of this technology includes neurofeedback training with meditation for stress- and/or anxiety-relief. This technology can also be used for some or all of the above applications under mixed mode that “intention” signals as well as human operations using hands or other parts of the body are used together to optimize the outcome of external devices.

### BRIEF DESCRIPTION OF THE SEVERAL VIEWS OF THE DRAWINGS

FIG. 1 is a diagram depicting the method according to one embodiment.

FIGS. 2A-2G are charts and diagrams showing the performance of one embodiment.

FIGS. 3A-3F compare CP vs DT training.

FIGS. 4A-4D show a similar comparison.

FIGS. 5A-5H demonstrate vertical control vs. horizontal control.

FIGS. 6A-6E depicts an example BCI system according to one embodiment.

FIGS. 7A-7C are example continuous pursuit trajectories.

FIGS. 8A-8B are Squared Tracking Correlation Histograms.

FIGS. 9A-9B show Continuous Pursuit vs. Discrete Trial BCI Learning.

FIGS. 10A-10B depict Influence of Eye Activity on BCI control.

FIGS. 11A-11B show Source vs. Sensor BCI Learning.

FIGS. 12A-12F show 2D CP Source vs. Sensor Spatial Threshold.

FIGS. 13A-13H show Online 1D Horizontal CP Source vs. Sensor BCI Performance.

FIGS. 14A-14H show Online 1D Vertical CP Source vs. Sensor BCI Performance.

FIGS. 15A-15C show Offline Source vs. Sensor Sensorimotor Modulation.

### DETAILED DESCRIPTION

Detecting mental intent and controlling external devices through brain-computer interface (BCI) technology has opened the doors to improving the lives of patients suffering from various neurological disorders, including amyotrophic lateral sclerosis and spinal cord injury. These realizations have enabled patients to communicate with attending clinicians and researchers in the laboratory by simply imagining actions of different body parts. While achievable task complexity varies between invasive and noninvasive systems, BCIs in both domains have restored once lost bodily functions that include independent ambulation, functional manipulations of the hands, and linguistic communication. As such, clinical interest is rapidly building for systems that allow patients to interact with their environment through autonomous neural control. Nevertheless, while technology targeting the restoration or augmentation of arm and hand control is of the highest priority in the intended patient populations, electroencephalography (EEG) based BCIs targeting such restorative interventions are some of the least effective. With exemplary clinical applications focusing on robotic- or orthosis-assisted hand control, it is paramount to improve upon the coordinated navigation of a robotic arm, as its precise positioning will be vital for the success of downstream actions. To meet this need, we present here a unified noninvasive framework for the continuous EEG-based 2-dimensional (2D) control of a physical robotic arm.

While BCI learning rates can vary among individuals, it is generally thought that a user's motivation and cognitive arousal play significant roles in the process of skill acquisition and eventual task performance. Although levels of internal motivation vary across populations and time, engaging users and maintaining attention via stimulating task paradigms may diminish these differences. Current BCI task paradigms overwhelmingly involve simple cued center-out tasks defined by discrete trials (DT) of neural control. While these tasks provide robust testbeds for novel decoding algorithms, they do not account for the random perturbations that invariably occur in daily life. Continuous analogues, in which users are not bound by time-limited objectives, enable control strategies that facilitate the extension of BCI towards the realistic control of physical devices in the home and clinic. Here, in order to produce robust robotic arm control that would be useful for daily life, we employed a continuous pursuit (CP) task in which users performed motor imagination to chase a randomly moving target. We found that CP task training produced stronger behavioral and physiological learning effects than traditional DT task training; an effect that can be credited to the Yerkes-Dodson law.

Poor signal quality can further complicate the ability to decode neural events, especially when utilizing noninvasive signals such as EEG. Spatial filtering has long been used to de-noise noninvasive BCI signals, and has recently offered promise in detecting increasingly diverse realistic commands. Electrical source imaging (ESI) is one such approach that uses the electrical properties and geometry of the head to mitigate the effects of volume conduction and estimate cortical activity. Dramatic improvements in offline neural decoding have been observed when using ESI compared to traditional sensor techniques; however, these approaches have yet to be validated online. By developing a real-time ESI platform, we were able to isolate and evaluate neural decoding in both the sensor and source domain without introducing the confounding online processing steps that often accompany other spatial filtering techniques (different classifiers, time windows, etc.).

In all, the framework presented here demonstrates a systematic approach to achieving continuous robotic arm control through the targeted improvement of both the user learning (“brain” component) and machine learning (“computer” component) elements of a BCI. Specifically, employing a CP task training paradigm increased BCI learning by nearly 60% for traditional DT tasks and by over 500% in the more realistic CP task. The utility of real-time ESI further introduced a significant 10% improvement in CP BCI control for users experienced in classical sensor-based BCI. Through the integration of these improvements, we demonstrated the continuous control of a robotic arm (Videos S4-7) at almost identical levels to that of virtual cursor control, highlighting the potential of noninvasive BCI to translate to real-world devices for practical tasks and eventual clinical applications.

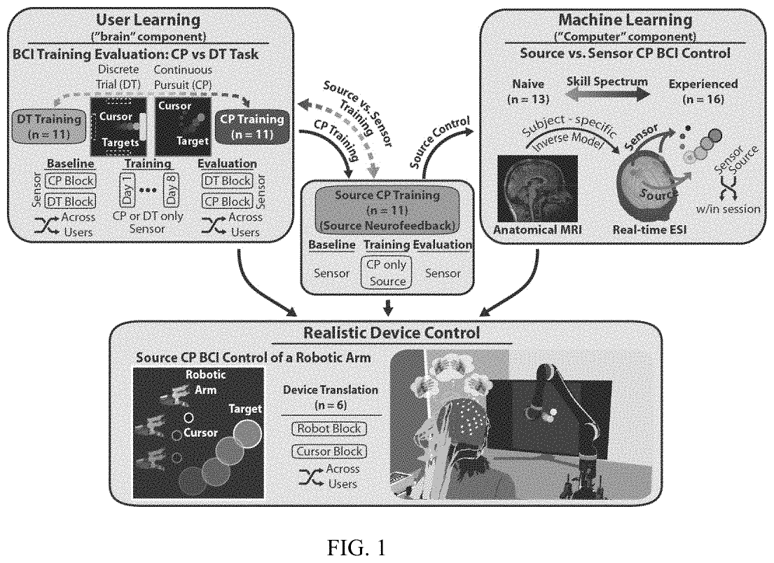

The online ESI-based decoding strategy described herein can be used for the continuous control of a robotic arm. However, the CP task and source signal approach should be thoroughly validated as useful training and control strategies, respectively (FIG. 1). Thirty-three individuals naïve to BCI participated in a virtual cursor BCI learning phase. The training length was set at ten sessions to facilitate practical data acquisition and to establish a threshold for future training applications. These thirty-three users were split into three groups, sensor domain CP training (CP), sensor domain DT training (DT), and source domain CP training (using real-time ESI, sCP). This design allowed us to answer: (1) which training task (CP vs. DT) and (2) which neurofeedback domain (source vs. sensor control) led to more effective BCI skill acquisition (see ‘Methods’ section for details on participant demographics and baseline group metrics). The within-session effects of source vs sensor control (virtual cursor) on CP BCI performance were tested on twenty-nine individuals, sixteen with prior BCI experience (sensor control) and thirteen naïve to BCI. Furthermore, six individuals with BCI experience (sensor DT cursor control) participated in experiments designed to compare the performance between virtual cursor and robotic arm control in a physically constrained variation of the CP task.

Noninvasive Continuous Virtual Target Tracking Via Motor Intent

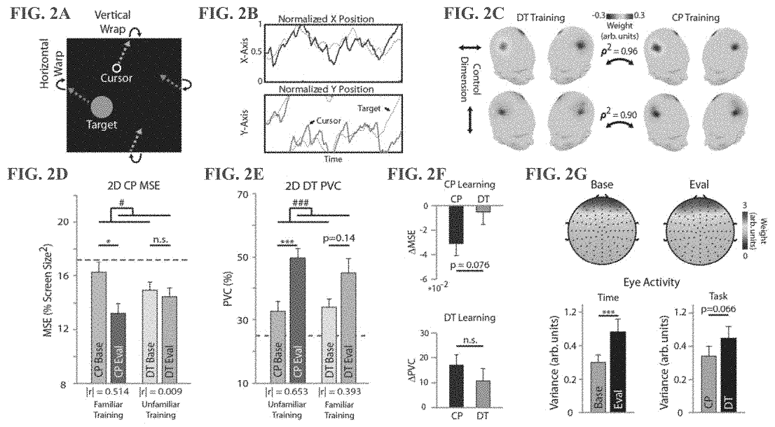

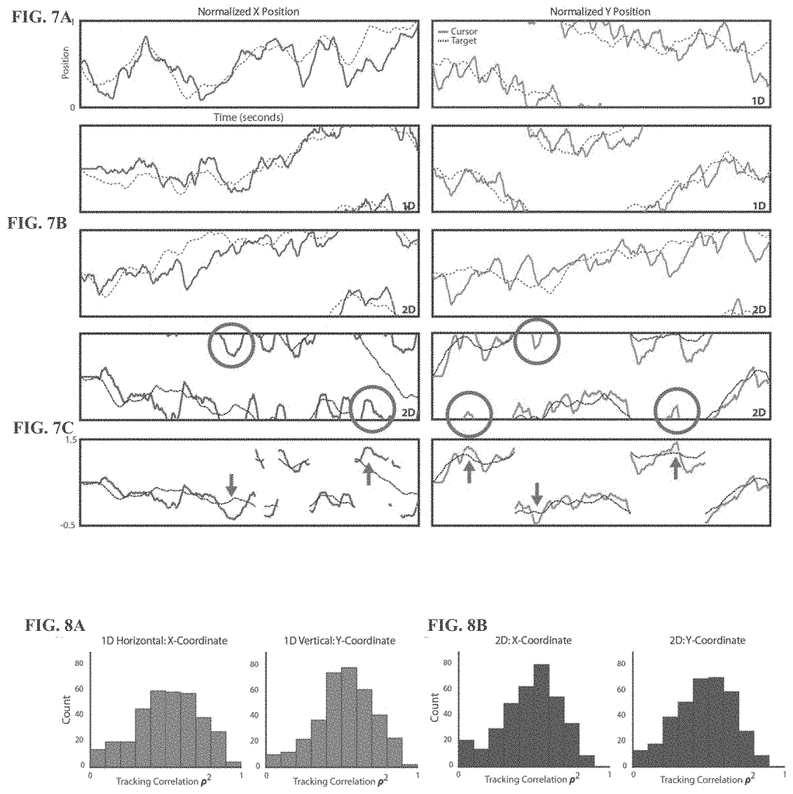

Throughout all experimental sessions, users were instructed to control the trajectory of a virtual cursor using motor imagination (MI) tasks; left- and right-hand MI for the corresponding left and right movement, and both hands MI and rest for up and down movement, respectively. These tasks were chosen based on previous cursor control and neurophysiological exploration. Horizontal and vertical cursor movements were controlled independently. CP trials lasted 60 seconds each and required users to track a randomly moving target within a square workspace (FIGS. 2A-2B and FIGS. 7A-7C). Previous implementations of similar tasks utilized technician controlled (manual) target trajectories, which can introduce inconsistencies and biases during tracking. To avoid such scenarios, target trajectories in the current work were governed by a Gaussian random process (see ‘Methods’ section). Nevertheless, it is possible for such a random process to drive the target towards stagnation at an edge/corner, which could synthetically distort performance. Therefore, to better estimate the difference between DT and CP task training, and contrary to previous work (18, 29), our initial CP task allowed the cursor and target to fluidly wrap from one side of the workspace to the other (top to bottom, left to right, and vice versa) upon crossing an edge (FIG. 2a-2b, FIGS. 7a-7b).

Trajectories from experienced users were unwrapped (FIG. 7C) to reveal squared tracking correlations of ρₕₒᵣ²=0.48±0.20 and ρᵥₑᵣ²=0.47±0.19.

Referring again to FIG. 1, which shows source-based continuous pursuit BCI robotic arm framework. The proposed framework addressed both the user and machine learning aspects of BCI technology before being implemented in the control of a realistic robotic device. User learning was addressed by investigating the behavioral and physiological effects of BCI training using sensor-level neurofeedback with a traditional discrete trial (DT) center-out task (n=11) and a more realistic continuous pursuit (CP) task (n=11) (top left). The effects of BCI training were further tested in the CP task using source-level neurofeedback (n=11) obtained through online electrical source imaging with user-specific anatomical models (center). This design allowed us to determine both the optimal task and neurofeedback domain for BCI skill acquisition. The machine learning aspect was further examined across the skill spectrum by testing the effects of source-level neurofeedback, compared to sensor-level neurofeedback, in naïve (n=13) and experienced (n=16) users in a randomized single-blinded design (top right). The user and machine learning components of the proposed framework were then combined to achieve real-time continuous source-based control of a robotic arm (n=6) (bottom). Comparing BCI performance of robotic arm and virtual cursor control demonstrated the ease of translating neural control of a virtual object to a realistic assistive device useful for clinical applications.

BCI Skill Acquisition and User Engagement

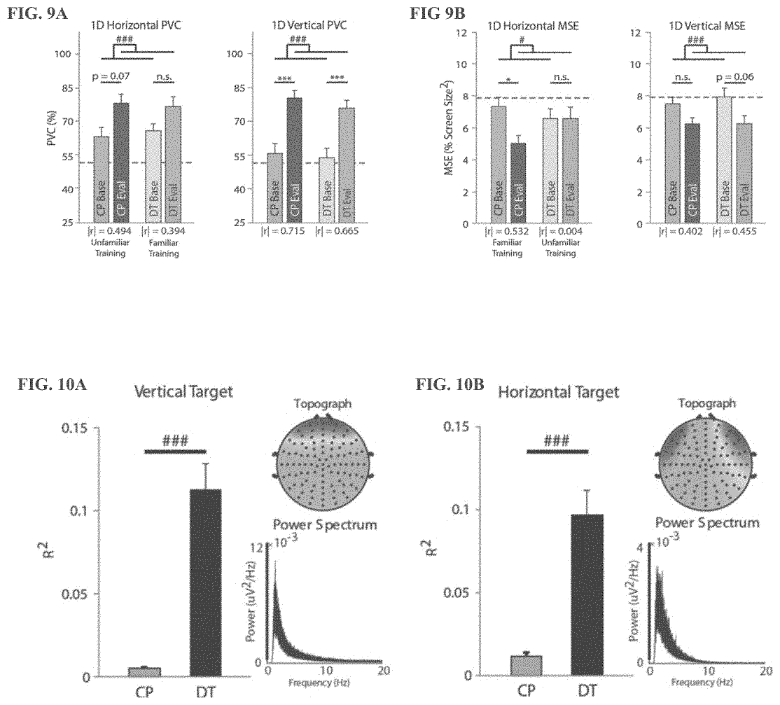

We investigated the utility of using the CP task for BCI skill acquisition in a pre-post study design by comparing BCI performance between populations trained by either the CP or DT task. Twenty-two individuals participated in a baseline session, eight training sessions, and an evaluation session. Baseline and evaluation sessions contained both DT and CP tasks (and MI without feedback) while training sessions contained only one task type, consistent throughout training according to each user's assigned group (DT or CP, n=11 per group, see ‘Methods’ section). All sessions for both groups utilized scalp sensor information. 1-dimensional (1D) horizontal DT performance was used to baseline match the two groups (FIG. 9A).

Electrodes used for online control were optimized on a session-by-session basis (see ‘Methods’ section), chosen from a set of 57 sensors covering the sensorimotor regions. Electrodes were identified for the horizontal and vertical control dimensions independently using the corresponding right vs. left hand MI and both hands MI vs. rest data sets. Throughout training, the two groups derived nearly identical feature (electrode) maps in the sensor domain containing focal bilateral scalp clusters overlying the cortical hand regions (FIG. 2C). These clusters were located and weighted in accordance with the underlying event-related (de)synchronization (ERD/S) generated during the corresponding MI tasks and are similar to those used in other noninvasive cursor control studies, identified through either data-driven or manual selection processes.

DT task performance was measured in terms of percent valid correct (PVC), computed as the number of hit trials divided by the total number of trials in which a final decision was made (valid trials). The corresponding CP task performance metric was mean squared error (MSE), i.e. the average normalized squared error between the target and cursor location over the course of a single run. Across these 22 participants, the results of a repeated-measures two-way ANOVA revealed a significant main effect of time for both the CP MSE (F(1,20)=7.39, p<0.05, FIG. 2d) and DT PVC (F(1,20)=19.80, p<0.005, FIG. 2E) metrics. To examine skill generalizability, we specifically considered the effects of training on the performance of familiar and unfamiliar tasks. Individuals trained with the CP task significantly improved in the same task after training (Tukey's HSD post hoc p<0.05), FIG. 2D, left bars), whereas those trained with the DT task did not (Tukey's HSD post hoc p=0.14, FIG. 2E, right bars). Previous work has indicated that DT task training can lead to strong learning effects (31), however, some users have required nearly 70 training sessions to do so (18). When considering unfamiliar tasks, the DT training group only modestly improved in the CP task after training (Tukey's HSD post hoc p=0.96, FIG. 2D, right bars) while the CP training group displayed a significant improvement in the DT task (Tukey's HSD post hoc p<0.005, FIG. 2E, left bars).

Since the two tasks varied greatly in control dynamics, it was difficult to draw comparisons between these differences. Therefore, in addition to statistical testing, we also examined the effect size (point biserial correlation, see ‘Methods’ section), a measure, unconfounded by sample size, of the magnitude of the difference within each performance metric between the baseline and evaluation sessions. Compared to the DT group, the effect sizes were far larger for the CP group for both tasks (FIGS. 2D-2E), displaying a 500% learning improvement in the CP task and a nearly 60% learning improvement in the DT task (FIG. 2F).

FIGS. 2A-2G 2 shows BCI Performance and User Engagement. FIG. 2A—Depiction of the CP edge wrapping feature. FIG. 2B—Tracking trajectory during an example 2D CP trial. FIG. 2C—Training feature maps for the DT and CP training groups for horizontal (top) and vertical (bottom) cursor control. ρ 2-squared correlation coefficient. FIGS. 2D-2E: 2D BCI performance for the CP (FIG. 2D) and DT (FIG. 2E) task at baseline and evaluation for the CP and DT training groups. The red dotted line indicates chance level. The effect size, |r|, is indicated under each pairs of bars. FIG. 2F—Task learning for the CP (top) and DT (bottom) tasks. FIG. 2G—Eye blink EEG component scalp topography (top) and activity (bottom left) at baseline and evaluation, and activity during each task (CP vs. DT) (bottom right). Bars indicate mean+standard error of the mean (SEM). Statistical analysis using a one- (F) or two-way repeated measures (D-E, G) ANOVA (n=11 per group) with main effects of task, and time and task, respectively. Main effect of time: #p<0.05, ###p<0.005. Tukey's HSD post hoc: * p<0.05, *** p<0.005.

To delineate the underlying physiology of these training differences, we investigated user engagement during both tasks by quantifying eye blink activity. Decreased blink activity has been implicated in heightened attentional processes and cognitive arousal during various tasks. These mental states can dramatically influence task training and performance; where stimulating tasks can facilitate skill acquisition, boring or frustrating tasks can inhibit performance. The eye blink component of the EEG was extracted during the baseline and evaluation sessions using independent component analysis (FIG. 2G, FIG. 10). Across all participants, blink activity was strongly dampened at the baseline (F(1,63)=9.84, p<0.005, FIG. 2G), suggesting heightened attention that was likely due to the novelty of BCI in general. Increased blink activity at the evaluation supports user skill acquisition, as less attention was required for improved performance. The large reduction in blink activity observed during the CP task, compared to the DT task (F(1,63)=3.51, p=0.066, FIG. 2G), suggests that the CP task elicited heightened user engagement during active control, a feature that may explain the more dramatic positive training effects.

Learning to Modulate Sensorimotor Rhythms

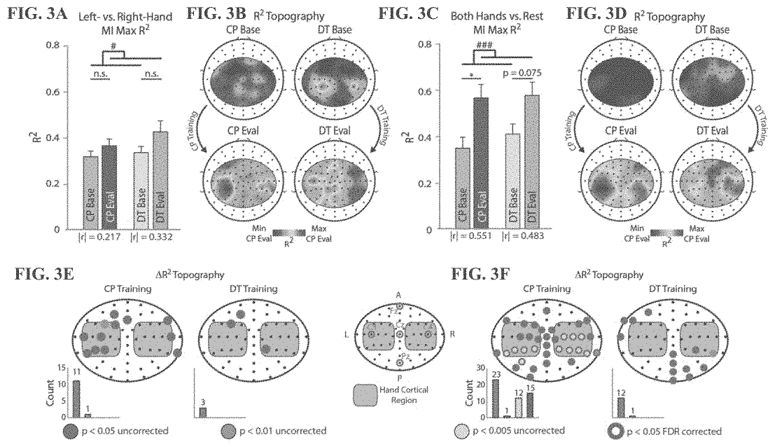

While BCI feedback plays a significant role in facilitating sensorimotor rhythm modulation, MI without feedback can provide a measure of a user's natural ability to produce the associated discriminative EEG patterns. Left- vs. right-hand MI (left vs. right) and both hands MI vs. rest (up vs. down) runs were analyzed individually. An index of modulation between any two mental states is represented as the regression output (R2) between the EEG alpha power and the task labels (see “Methods' section). Only the 57 sensorimotor electrodes used for online control were included in this analysis. While sensorimotor modulation significantly increased for both task pairs from baseline to evaluation (horizontal F(1,20)=4.70, p<0.05, vertical F(1,20)=21.01, p<0.005; FIGS. 3A, 3C), the spatial distribution of these improvements are more meaningful in evaluating the effectiveness of BCI training. Except for mild baseline modulation in the DT group, no strong patterns were apparent for either task pair prior to training. For the horizontal dimension at the evaluation session, the CP group produced highly focal bilateral modulation patterns, whereas more global modulation was observed for the DT group (FIG. 3B). Evaluation topographies were more consistent between the two training groups for the vertical dimension (FIG. 3D). Electrodes displaying a significant improvement in modulation were far more numerous for the CP group than for the DT group for both horizontal (CP: 12, DT: 3; FIG. 3E) and vertical (CP: 37, DT: 13; FIG. 3F) tasks. Furthermore, these significant electrodes cluster far closer to scalp regions covering the approximate hand cortical regions (e.g. C3-4, CP3-4, etc.) in the CP group.

These localized changes provide compelling evidence that the enhanced behavioral improvement seen in the CP training group was accompanied by consistent physiological changes in sensorimotor modulation (R2 values).

FIGS. 3A-3F shows Electrophysiological Learning Effects. FIGS. 3A-3B: Left vs. right MI task analysis. (A) Maximum sensorimotor R2 value for the CP and DT training groups for horizontal control task. The effect size, |r|, is indicated under each pair of bars. FIG. 3B—R2 topographies at baseline (top row) and evaluation (bottom row) for the CP and DT training groups for horizontal control tasks. FIGS. 3C-3D: Both hands vs. rest MI task analysis. Same as FIGS. 3A-3B for vertical control tasks. FIGS. 3E-3F: Statistical topographies indicating electrodes that displayed a significant increase in R2 values for the horizontal (FIG. 3E) and vertical (FIG. 3F) control tasks. The electrode map in the middle provides a reference for the electrodes shown. Bar graphs below each topography provide a count for the number of electrodes meeting the various significance thresholds. Bars indicate mean+SEM. Statistical analysis using a one- (FIG. 3E-3F) or two-way repeated measures (FIG. 3A, FIG. 3C) ANOVA (n=11 per group) with main effects of time (blue—p<0.05, green—p<0.01, yellow—p<0.005, red outline—p<0.05 false discovery rate corrected), and time (#p<0.05, ###p<0.005) and training task, respectively. Tukey's HSD post hoc: * p<0.05.

Source Neurofeedback does not Further Facilitate CP BCI Learning

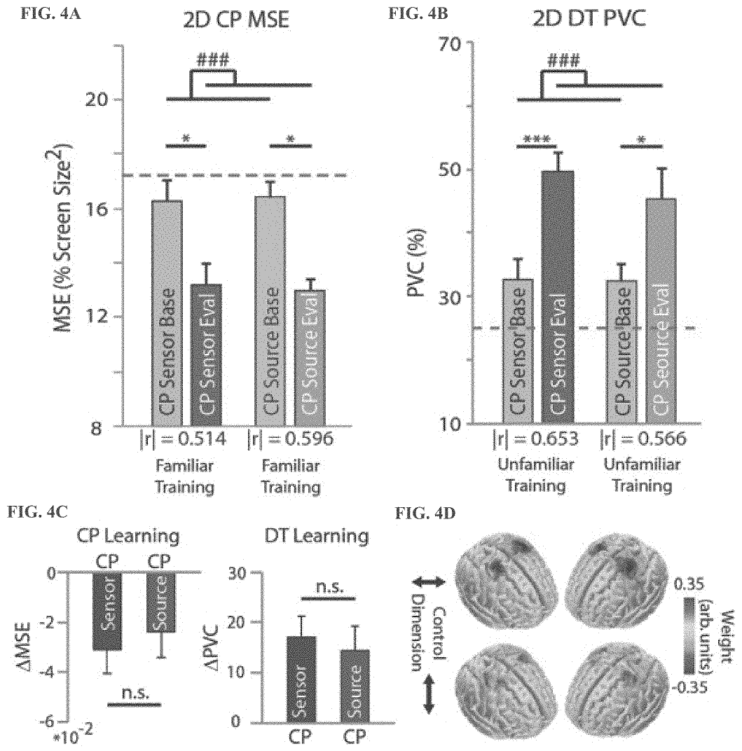

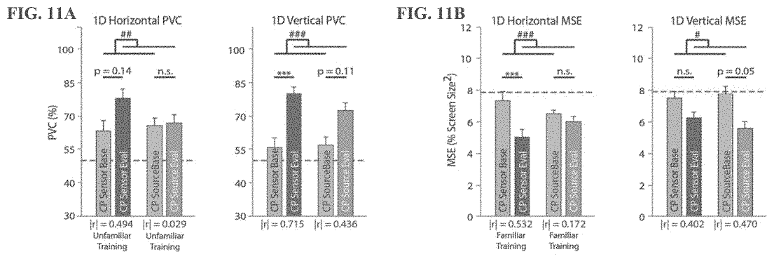

While the CP task allowed us to target user learning and progress towards the robust online control of a robotic arm, we additionally wanted to address the machine learning element. To evaluate whether real-time ESI-based decoding improved performance throughout training, we recruited an additional group of BCI naïve individuals (n=11) for CP training using source neurofeedback (source control, sCP). This sCP group was baseline matched to the previous CP (and DT) group (sensor control) (FIG. 11A). For source control, we implemented user- and session-specific inverse models into the online decoding pipeline for the CP task. Similar to the CP group, the sCP group significantly improved in both the 2D CP (Tukey's HSD post hoc p<0.05, FIG. 4A, right bars) and 2D DT tasks (Tukey's HSD post hoc p<0.05, FIG. 4B, right bars) after training. Accordingly, very similar learning effects were observed for both tasks in the CP and sCP groups (FIG. 4C). The final performance and learning rates were consistent between the two training groups (CP and sCP), supporting the groups' shared familiar and unfamiliar task proficiency.

Feature selection in the source domain identified distinct cortical clusters, optimized through anatomical and functional constraints, for online control and were selected on a session-by-session basis (see ‘Methods’ section). As expected, sCP training feature maps highlighted hand cortical regions for both control dimensions throughout training (FIG. 4D). It should be noted that the baseline and evaluation sessions for the sCP group were completed in the sensor domain to maintain consistent conditions with the other training groups. While training duration was fixed at eight sessions with no intermediary testing, further investigation at different stages of learning may help pinpoint when source-based decoding may benefit BCI skill acquisition.

FIGS. 4A-4D. Source-Level Neurofeedback. FIGS. 4A-4B: 2D BCI performance for the CP (FIG. 4A) and DT (FIG. 4B) task at baseline and evaluation for the CP and source CP (sCP) training groups. The red dotted line indicates chance level. The effect size, |r|, is indicated under each pairs of bars. FIG. 4C—Task learning for the CP (left) and DT (right) tasks. Bars indicate mean+SEM. Statistical analysis using a one- (FIG. 4C) or two-way repeated measures (FIG. 4A-4B) ANOVA (n=11 per group) with main effects of training decoding domain, and time and training decoding domain, respectively. Main effect of time: #p<0.05, ###p<0.005. Tukey's HSD post hoc: * p<0.05, *** p<0.005. FIG. 4D Group-level training feature maps for the training groups for horizontal (top) and vertical (bottom) cursor control. User-specific features were projected onto a template brain for group averaging.

EEG Source Imaging Enhances Neural Control in Defined Skill States

To thoroughly investigate the effects of source control (real-time ESI) on CP task performance (and potential future benefits for robotic arm control), we performed within-session comparisons of source and sensor virtual cursor control on users in stable skill states. The CP task was chosen for further analysis because it is more applicable to robotic arm control than the DT task and displayed both increased difficulty and skill acquisition. Our investigation included both extremes of the BCI skill spectrum; experienced users (12.8±8.9 hours of prior BCI training, n=16) participated in up to three sessions and naïve users (no prior BCI training, n=13) participated in a single session (to avoid confounding effects of early learning in >1 session). User- and session-specific inverse models were also utilized for these participants.

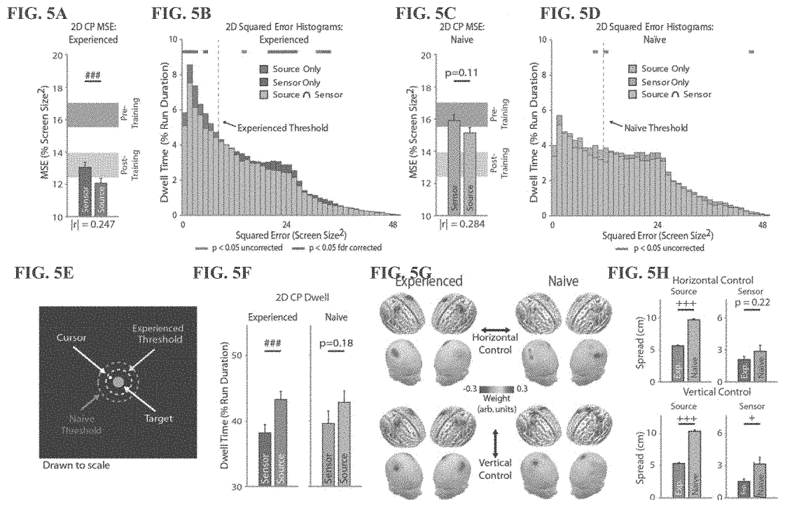

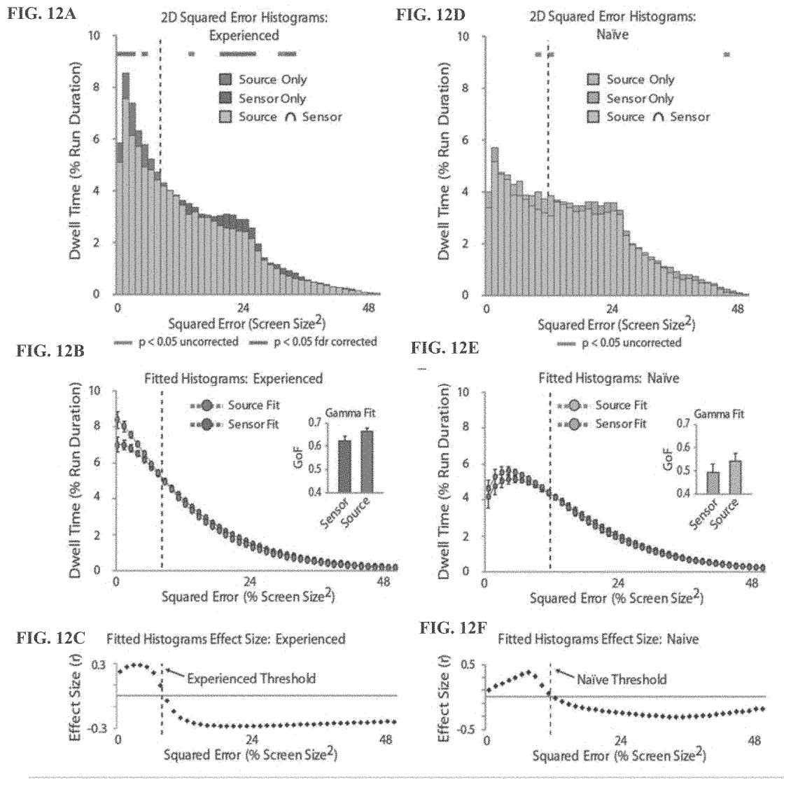

For experienced users, source control improved performance over that of conventional sensor control, producing a significant reduction in the 2D MSE (F(1,69)=9.83, p<0.01, FIG. 5A). Unsurprisingly, the sensor and source MSE values clustered near those of the CP training group post-training (evaluation), reinforcing their skilled state. The spatial extent of the observed improvement in the CP task was characterized through squared error histograms (FIG. 5B), with source values shifting toward smaller errors and sensor values shifting toward larger errors. By fitting gamma functions to these histograms, we derived a quantitative threshold, independent of cursor/target size, for statistically testing the spatial extent of the performance difference (FIG. 12). Experienced users dwelt within this defined region, a disc with a diameter of 16.67% of the workspace width centered on the target (FIG. 5E), for significantly more time during source control than sensor control (F(1,69)=20.96, p<0.005, FIG. 5F).

Naïve users also demonstrated overall improved online performance with source control, although this improvement did not reach significance for 2D control (F(1,12)=3.02, p=0.11, FIG. 5C). Nevertheless, the effect size for the performance difference was strikingly similar to that of experienced users (FIGS. 5A,5c, Table S1), indicating an improvement of similar magnitude. As expected, the sensor and source control MSE values for the naïve users were comparable to those of the CP training group pre-training (baseline, also naïve). This consistency, independent of skill level, highlights a robust positive influence of source control on online performance. Furthermore, the squared error histograms (FIG. 5D) and extent threshold measures for naïve users (FIG. 5E) displayed analogous trends to those of experienced users, however, these did not reach significance (F(1,12)=2.02, p=0.18, FIG. 5F).

FIGS. 5A-5H. Online 2D CP Source vs. Sensor BCI Performance. FIG. 5A-5B: Experienced user performance (n=16). FIG. 5A Group-level MSE for source and sensor 2D CP cursor control. Light and dark gray blocks represent performance for the CP training group (n=11, FIG. 2D) before (naïve) and after training (experienced). The effect size, |r| is indicated under the pair of bars. FIG. 5B: Group-level squared-error histograms for 2D CP sensor and source cursor control. FIG. 5C-5D: Naïve user performance (n=13). Same as FIG. 5A-5B for naïve user data. FIG. 5E: Scale drawing of the continuous pursuit paradigm workspace displaying the spatial threshold derived from for experienced (yellow) naïve (green) user data (Fig. S6). FIG. 5F: Cursor dwell time within the spatial threshold for experienced (left) and naïve (right) users. FIG. 5G: Group-level feature maps for horizontal (top) and vertical (bottom) cursor control for naïve (left) and experienced (right) users. User-specific features were projected onto a template brain for group averaging. FIG. 5H: Feature spread analysis between experienced and naïve users for source (left) and sensor (right) features for horizontal (top) and vertical (bottom) control. Bars indicate mean+SEM. Statistical analysis using a one- (FIG. 5C-5D) or two-way repeated measures (FIG. 5A-5B) ANOVA with main effects of decoding domain, and time and decoding domain, respectively. Main effect of decoding domain: ###p<0.005 (FIG. 5A, 5C, 5F), gray bar p<0.05 uncorrected, red bar p<0.05 false discovery rate corrected (FIG. 5B, 5D). Mann-Whitney U test with Bonferroni correction for multiple comparisons (H): +p<0.05, +++p<0.005.

When looking at the feature maps (FIG. 5G), an important dichotomy can be observed between naïve (weak, sporadic clusters) and experienced (strong, focal clusters) users for both control dimensions that parallels the trends previously observed in the modulation index topographies before (low, sporadic modulation) and after (high, focal modulation) training (FIG. 3B, 3D). To quantify the focality/diffuseness of these features, we computed the spread of the group-level feature maps (FIG. 5H), defined as the average weighted distance between the feature location and the hand knob (source space) or C3/C4 electrode (sensor space) (see ‘Methods’ section). We observed both significant or near significant reductions in the feature spread for experienced users, compared to naïve users, in both the horizontal (Mann-Whitney U test with Bonferroni correction, source: p<0.005, sensor: p<0.05) and vertical (Mann-Whitney U test with Bonferroni correction, source: p<0.005, sensor: p=0.22) control dimensions. This physiological difference between naïve and experienced users is in line with their performance difference (MSE) and further supports the contrast in BCI proficiency among the two groups and the overarching effect of source-based control depending on user skill level.

Source-Based CP BCI Control of a Robotic Arm

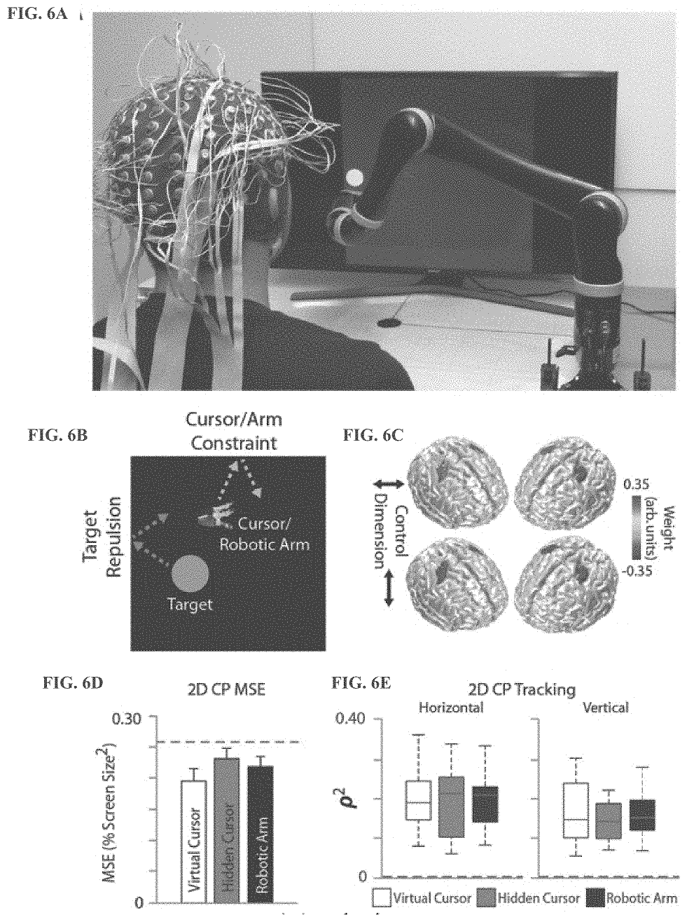

Having robustly validated our proposed BCI framework in a controlled environment, we completed our study by transitioning to the applied physical source control of a robotic arm (FIG. 6A). Although the cursor and target wrapping allowed for more complicated control strategies and scenarios, such a feature could not exist in a real-world setting. Therefore, we implemented a modified form of the CP task in a robotic arm control paradigm, where the edge wrapping feature was replaced with an edge repulsing feature (FIG. 6B). Six experienced users (8.3±2.9 hours of previous BCI training) participated in five source CP BCI sessions containing both virtual cursor and robotic arm control, block-randomized across individuals and sessions. As no paradigm was implemented to determine performance values before and after training in the modified task, participants were screened for experience and skill level beforehand (see ‘Methods’ section). Physiological support for user skill level was additionally observed in the group-level feature maps (FIG. 6C) that displayed comparable characteristics to those of other experienced users participating in this study (FIG. 5G).

When users were directly controlling the robotic arm, the behavior of a hidden virtual cursor was also recorded to ensure proper mapping of the arm position in physical space. Across all sessions and individuals, median squared tracking correlation values reached ρₕₒᵣ²=0.13 (1QR=0.04−0.32) and pᵥₑᵣ²=0.09 (1QR=0.03−0.28) in the horizontal and vertical dimensions, respectively, for 2D control. In transitioning between virtual cursor and robotic arm control, we observed similar MSE values among the three tracking conditions; virtual cursor, hidden cursor, and robotic arm (F(2,40)=2.62, p=0.086, FIG. 6d), indicating a smooth transition from the control of a virtual object to a real-world device. This likeness in control quality was further revealed through a lack of significant difference in the squared tracking correlation (ρ2) for both the horizontal (F(2,40)=0.13, p=0.88, FIG. 6e) and vertical (F(2,40)=0.77, p=0.47, FIG. 6e) dimensions. Tracking performance was significantly greater than chance for all control conditions and dimensions (Mann-Whitney U test with Bonferroni correction, all p<0.05). Overall, the striking similarity between virtual cursor control and robotic arm control highlights the possibility of integrating virtual cursor exposure into future clinical training paradigms where patients have limited access to robotic arm training time.

FIGS. 6A-6E. Source-Based CP BCI Robotic Arm Control. FIG. 6A: Robotic arm CP BCI setup. Users controlled the 2D continuous movement of a seven degree-of-freedom robotic arm to track a randomly moving target on a computer screen. FIG. 6B: Depiction of the CP edge repulsion feature (in contrast to the edge wrapping feature—FIG. 2A) utilized to accommodate the physical limitations of the robotic arm. FIG. 6C: Group-level feature maps for the horizontal (top row) and vertical (bottom row) control dimensions projected onto a template brain. FIG. 6D: Group-level 2D MSE for the various control conditions. Bars indicate mean+SEM. FIG. 6E: Box-and-whisker plots for the group-level squared tracking correlation (ρ2) values for the horizontal (left) and vertical (right) dimensions during 2D CP control for the various control conditions. The blue line indicates the median, the top and bottom of the box the 25th and 75 percentiles, respectively, and the top and bottom whiskers the respective min and max values. Control conditions include virtual cursor (white), hidden cursor (gray), and robotic arm (black). The red dotted line indicates chance level. Statistical analysis using a repeated measures two-way ANOVA (n=6 per condition) with main effects of time and control condition.

Discussion

The research presented here describes an encompassing approach aimed at driving noninvasive neural control towards the realistic daily use of a robotic device. We have demonstrated that the CP BCI paradigm can not only be used to successfully gauge a user's BCI proficiency, but can also serve as a more effective training tool than traditional center-out DT tasks, accelerating the acquisition of neural cursor control and driving the associated physiological changes. Contrary to users trained with the DT task, those trained with the CP task displayed significant performance improvements in familiar and unfamiliar tasks (FIG. 2d-f), demonstrating highly flexible skill acquisition. These results were further supported in a third group that also trained with the CP task (FIG. 4a-c). Participants in this group (sCP), displayed nearly identical learning effects as the original sensor CP group, while training with source control, providing confidence for the reproducibility of the effects of CP task training.

As training progressed, it became apparent that the strategies developed by users differed significantly depending on the training task. For example, various individuals in the DT training group reported utilizing strategies involving selectively attending to their hand/s through peripheral vision without necessarily focusing on the cursor position. While such strategies were effective for DT tasks, users employing them often struggled with the CP tasks in the evaluation session, as the moving target and cursor required constant visual attention and adjustment of motor-related mental intent. In this sense, many of these users somewhat ignored the feedback when training with the DT task and treated it similarly to the MI without feedback, reducing its effectiveness.

The lower success of such strategies manifested within the MI EEG of the DT group as sporadic patterns of modulation after training (FIG. 3d) which is also consistent with the lower levels of cognitive arousal observed during the traditional DT task, compared to the CP task (FIG. 2g). We believe that the target dynamics and screen wrapping feature of the CP task (FIG. 2a) likely perturb fluid target tracking and require heightened attention during cursor control. These conclusions support the overarching concept of integrating human factors, such as virtual reality techniques (34, 35), into cognitive-based training tools for improving both user engagement and task performance (20, 36-38), and should be considered in future generations of BCIs.

Seminal works implementing similar continuous tracking tasks using invasively acquired signals reported comparable squared tracking correlation values over a decade ago (29). While the field of invasive neural decoding has surpassed these benchmark results to include high degree-of-freedom and anthropomorphically functional tasks, qualitative similarities can be seen between these two modalities. In accordance with invasive reports, users in our study struggled to keep the cursor in a single location, often exhibiting oscillatory tracking behavior around the target (FIG. 2b, FIGS. 7A-7B). While these actions demonstrate directed cursor trajectories towards the target and highlight the ability of our system to accurately capture the users' dynamic mental intent, the tracking correlation is effectively reduced and may benefit from more advanced decoding methods.

It has been argued that motor neurons encode cursor velocity during neural cursor control, with numerous decoding algorithms utilizing such properties to drastically improve user performance over classical techniques. In particular, modeling neuronal behavior as a dynamical system has recently yielded significantly improved online decoding results and may provide even more complex and efficient device control in upcoming invasive and non-invasive work. This decoding strategy would be particularly attractive to neural control in the CP task presented here, given the clear analogue of our control output to under-dampened control dynamics. While this information would be valuable to reduce or eliminate the previously described cursor oscillations, it has yet to be observed if these details can be detected via scalp recordings. Nevertheless, noninvasive neural signals have recently been shown to contain information encoded on the spatial scale of cortical columns (sub-mm), indicating the ability to decode neural activity with very fine spatial-temporal resolution from outside the skull.

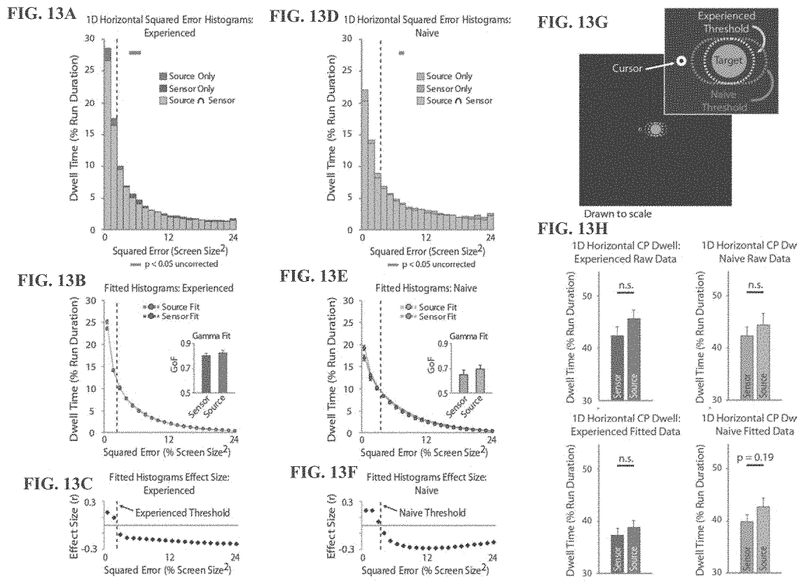

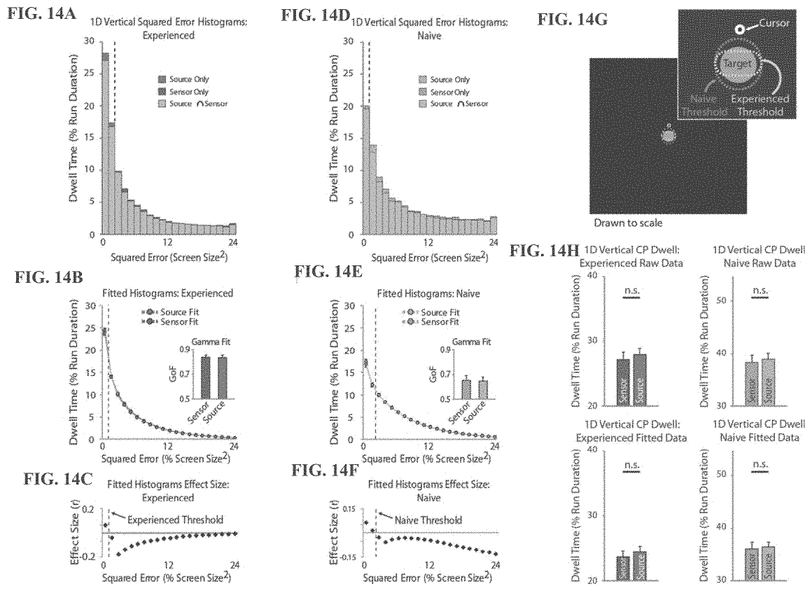

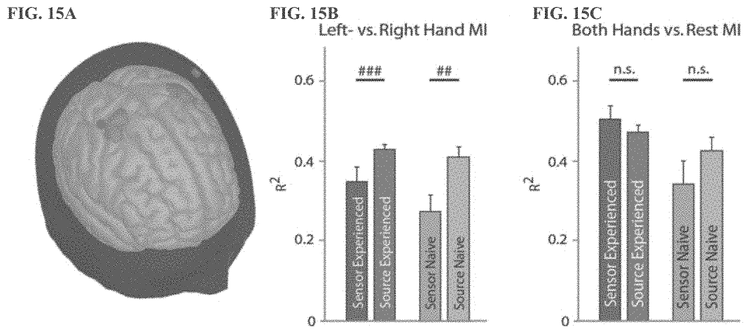

Over the past few decades, the reconstruction of cortical activity through ESI has exemplified the push to increase the spatial specificity of noninvasive recordings and has been shown to provide superior neural decoding when compared to scalp sensor information. Similar to these previous works, we found that, in general, source features were more correlated with cued motor-related mental states than sensor features (FIG. 15). Furthermore, in closed-loop CP BCI control, we found that the inclusion of online ESI improved performance in naïve and experienced users, consistent with offline enhancements (FIG. 5, FIGS. 13-15). The increased task-specific source modulation indicates a higher sensitivity for detecting changes in a user's motor-related mental state and is likely a product of the principles of ESI and its use in modeling and counteracting volume conduction. CP cursor control requires highly dynamic cognitive processes to recognize and correct for the random and sudden changes in the target's trajectory during tracking. We therefore hypothesize that the fast, real-time control required during the CP paradigm takes advantage of the heightened sensitivity of ESI modulation, allowing for quicker responses that more accurately resemble the dynamics involved in the CP task. This phenomenon was apparent during the within-session comparisons of source and sensor control (FIG. 5, FIGS. 13-14); however, it is possible that with sufficient training, the feedback domain becomes less important for skill acquisition (FIG. 4).

We feel it is necessary to acknowledge the decline in performance that occurred between the original CP task and the modified CP task which we believe to be strongly attributed to the task modifications made for the physical constraints of the robotic arm. The presence of the physical robotic device inherently creates a more distracting environment for neural control compared to that of a virtual cursor. We found that with the robotic arm mounted on the right side of the users (FIG. 1 bottom, FIG. 6a), visual obstruction of the target was common when the arm was directed to reach across the user to the left side of the screen, often perturbing target tracking. Additionally, while participants here displayed previous BCI proficiency, they had less experience than those participating in the original CP task validation. We believe that this combination of reduced user experience and enhanced sensory loading caused by the more complex human-device interaction involving the robotic arm led to a reduction in performance compared to the highly controlled virtual cursor control environment.

The results presented here demonstrate that CP control provides a unique opportunity for the complex control of a virtual cursor and robotic device, without requiring discretized, prolonged task sequences that can make even simple task completion long and frustrating. Users were able to smoothly transition between virtual cursor and robotic arm control with minimal changes in performance (FIG. 6d-e), indicating the potential ease of integrating such a noninvasive assistive tool into clinical applications for autonomous use in daily life. It should be noted that invasive systems have already demonstrated a level of control similar to such a noninvasive hypothetical; however, while such invasive approaches may offer much-needed help to a restricted number of patients with severe physical dysfunctions, the majority of impaired persons will likely not qualify for participation due to both medical and financial limitations. Additionally, it is apparent from previous work that access to sufficiently large patient populations for concrete and statistically significant conclusions may be difficult to obtain. Therefore, there is a strong need to further develop noninvasive BCI technology so that it can benefit the majority of patients and even the general population in the future. The effective training paradigm and additional ESI-based performance improvement demonstrated here, as well as the integration of such targeted enhancements towards robotic arm control, offer increasing confidence that noninvasive BCIs may be able to expand to widespread clinical investigation. In fact, we observed that for robotic arm control, generic head models, rather than those derived from user-specific MRIs, were sufficient for high quality performance (see ‘Methods’ section). Therefore, in all, the work presented in this paper is necessary for current EEG-based BCI paradigms to achieve useful and effective noninvasive robotic device control and its results are pertinent in directing both ongoing and future studies.

Materials and Methods:

Brain-Computer Interface Tasks

Motor Imagery w/o Feedback

EEG data during Motor imagery (MI) without feedback was collected at the beginning of each session, one run for left- vs right-hand MI and one for the both hands MI vs rest. Each run consisted of 10 randomly presented trials per task. Each trial consisted of three seconds of rest followed by four seconds of a visually cued MI task.

Discrete Trial Task

The discrete trial (DT) paradigm was composed of fixed target locations and center-out intended cursor trajectories. This paradigm consisted of 21 trials, with targets presented in a random order. Each trial began with a three second rest period, followed by a two second preparation period in which the target was presented to the user. Users were then given up to six seconds to move the cursor to hit the target. A one second inter-trial interval bridged two adjacent trials. Feedback (cursor movement) was not provided during the first trial to calibrate the normalizer as described in the Online Signal Processing section.

During baseline and evaluation sessions trials ended upon either a collision with a target or after 6 seconds with no collision. During training sessions, each trial lasted a full 6 seconds, requiring users to maintain their cursor over the target location for as long as possible within a boundary-constrained workspace. In this sense, during training, each DT run contained 120 seconds of online BCI control, consistent with the 120 second continuous pursuit runs

Continuous Pursuit Task

The continuous pursuit (CP) stimulus paradigm was implemented using custom Python scripts in the BCPy2000 application module of BCI2000 (47). This paradigm involved the continuous tracking of a target; each run was comprised of two 60 s trials separated by a one-second inter-trial interval. To produce smoothly varying random target movement, the position of the target was updated in each frame using a simple kinematic model. Random motion was obtained by applying a randomly generated one- or two-dimensional external force F^{\vec{ }}.EF, as in Eq. 1, drawn from a zero-mean fixed-variance normal distribution.

\vec{F}ₑₓₜ˜ [symbol] ₂(0,σ²) Eq. 1

To effectively limit maximum target velocity, a friction force and drag force F^{\vec{ }}K were also applied. The friction and drag forces are represented in Eq. 2 and Eq. 3 respectively, where μ indicates the coefficient of friction, δ the drag, and v^{\hat{ }}(F) the velocity of the cursor at time step t. Here, ∥ ∥ denotes the Euclidian norm

$$\begin{matrix}{\vec{F}}_{f} = - μ \frac{{\vec{v}}^{( t )}}{{‖ {\vec{v}}^{( t )} ‖}_{2}} & Eq . 2 \\ {\vec{F}}_{v} = - δ {\vec{v}}^{( t )} {‖ {\vec{v}}^{( t )} ‖}_{2} & Eq . 3\end{matrix}$$

When divided by the arbitrary target mass m, the combination of these forces represents the total instantaneous acceleration of the target. Integrating with respect to time, as noted in Eq. 4, produces the updated target velocity v^{\vec{ }}(t+1) at the new time point.

$$\text{?} = \text{?} + \frac{\text{?}}{\text{?}} \text{?} \\ \text{?} \text{indicates text missing or illegible when filed} \qquad Eq . 4$$

For the Training and Source vs. Sensor experiments described in subsequent sections, the cursor and target were allowed to wrap from one side of the workspace to the other (left to right, top to bottom, and vice versa). Contrary to this, for the Robotic Arm vs. Virtual Cursor experiments, the target was repelled by the edges of the workspace to make the task more realistic and accommodate the physical limitations of the robotic arm. Repulsion was accomplished by inverting all applied forces that would push the target continuously into a wall, while still randomly generating magnitudes and directions for irrelevant forces. Unlike the target dynamics, the cursor and robotic arm could press against the edge of the bounded region given the appropriate force vector.

Noise/Chance Performance Estimation

Chance performance in the CP paradigm was estimated by collecting 15 (standard) or 70 (physically constrained) data sets each for 1-dimensional (1D) horizontal (LR), 1D vertical (UD), and 2-dimensional (2D) control tasks with the electrode sets plugged in, but not connected to a human scalp. Chance performance in the DT paradigm was determined by dividing 100% by the number of targets in each control dimension. This is valid as trials which time out are typically excluded when calculating performance for the DT task.

Experimental Design

68 healthy humans were informed and participated in different phases of this study after providing written consent to a protocol approved by the relevant Institutional Review Board at the University of Minnesota or Carnegie Mellon University.

Training

33 individuals (average age: 24.8±10.6 yrs., 30 right-handed, 18 male) naïve to BCI participated in longitudinal BCI training over the course of 10 experimental sessions that included one baseline session, eight training sessions, and one evaluation session. Participants were tested on all tasks at the baseline and evaluation time points to assess training effectiveness, completing one block of DT tasks and one block of CP tasks, block wise randomized across individuals. The blocks for each paradigm were composed of two runs of 1D LR, 1D UD, and 2D control. Participants were divided into three training groups using the 1D LR DT performance as the balancing metric (Fig. S3a, Fig. S5a). Naïve participants obtaining percent valid correct (PVC) values of >80% for both runs of any of the three DT dimensions were excluded from the training cohort as these users are often considered proficient (n=5/38) (14, 28). Participants underwent eight training sessions at 12 runs per session, with only their specified task paradigm; DT sensor, CP sensor, or CP source.

These eight training sessions were broken into 2×1D LR, 2×1D UD, and 4×2D control to progress towards more difficult tasks near the end of training. The evaluation session was identical to the baseline session, again with the task block order randomized across individuals. Baseline and evaluation sessions were all completed using sensor control for consistency across groups. Participants underwent 2-3 sessions per week with an average inter-session interval of 3.69±2.99 days.

Source vs. Sensor

29 individuals participated in experiments testing the within-session effects of source vs sensor control on the CP BCI task. 16 users (average age: 22.67±8.1 yrs., 15 right handed, 6 male) with an average of 12.8±8.9 hours of prior BCI experience and 13 users (average age: 21.8±5.0 yrs., 12 right handed, 8 male) naïve to BCI participated in this portion of the study. Experienced users participated in up to three BCI sessions and the naïve users in a single session to avoid the confounding effects of learning. There were no exclusion criteria in this phase of the study as participants were in well-defined naïve or experienced states. A user-specific anatomical Mill was collected for each individual according to the Mill Acquisition section. In each BCI session, participants completed 12 runs of CP BCI (4×1D LR, 4×1D UD, and 4×2D) with the decoding strategy (sensor or source) being randomized and balanced across the population.

Robotic Arm vs. Virtual Cursor

6 individuals (average age: 25.2±6.5 yrs., 5 right handed, 3 male, 8.3±2.9 hours of previous BCI training) participated in experiments comparing virtual cursor and robotic arm control. Participants for this phase were screened using sensor-based 1D and 2D DT tasks using the BCI2000 AR alpha (8-13 Hz) power estimation of C3 and C4, spatially filtered with the local pseudo-Laplacian using a Neuroscan Synamps2 (Compumedics Ltd., Victoria, Australia) 64-channel system. Participants were excluded based on a two-stage performance evaluation: (1) failure to achieve >70% 1D PVC (sessions 1-2) or >40% 2D PVC (session 2) in two sequential runs, and (2) failure to achieve >90% 1D PVC and >70% 2D PVC (sessions 3-5) in two sequential runs. Six of nineteen recruited participants passed these criteria.

All robotic arm experiments were conducted on a Samsung 43 in 4K television, allowing large, practical workspaces for both the robotic arm and the virtual cursor. Each user participated in five source CP BCI sessions containing 12 runs (60 s) (session 1-2: 6×1D LR, 6×1D UD; session 3-5: 3×1D LR, 3×1D UD, 6×2D) of both virtual cursor and robotic arm control in block-randomized order across users. Some users were asked to return for a sixth session to record video of continuous robotic arm and virtual cursor control. Robotic arm endpoint locations were mapped 1:1 to cursor positions on the screen, with inverse kinematics employed to solve for optimal joint angles and arm trajectories. The robotic arm workspace was square with a 0.48 m side length. All robotic arm control was conducted using the Kinova Jaco Assistive Robotic Arm with a 3-finger attached gripper.

For all BCI sessions, participants were seated in a padded chair approximately 90 cm from a computer screen. Unless otherwise stated, users were fitted with a 128-channel BioSemi (BioSemi, Amsterdam, The Netherlands) EEG headcap of appropriate size and positioned according to the international 10-20 system. EEG was recorded at 1024 Hz using an ActiveTwo amplifier with active electrodes (BioSemi, Amsterdam, The Netherlands).

MRI Acquisition

User-specific anatomical MRI images were acquired on a 3T MRI machine (Siemens Prisma, Erlangen, Germany) using a 32-channel head coil. High resolution (1 mm isotropic) anatomical images were acquired for each participant using a T1-weighted magnetization prepared rapid acquisition gradient echo (MP-RAGE) sequence (TR/TE=2350 ms/3.65 ms, FA=7o, TA=05:06 min, R=2 acceleration, matrix size: 256×256, FOV: 256×256).

Frequency-Domain Electrical Source Imaging (FDESI)

For the Source vs. Sensor experiments, the anatomical MRI from each user was segmented in FreeSurfer and uploaded into the MATLAB-based Brainstorm toolbox. For the Robotic Arm vs. Virtual Cursor experiments, the Colin27 template brain was used for all users. The cortex was downsampled to a tessellated mesh of ˜15000 surface vertices and broken into 12 bilateral regions based on the Destrieux atlas. A central region of interest (ROI), composed of various sensorimotor areas (Table S2), was utilized for feature extraction and online source control.

At the beginning of each BCI session in the Source vs. Sensor experiments, EEG electrode locations were recorded using a FASTRAK digitizer (Polhemus, Colchester, Vt.) using the Brainstorm toolbox. Electrode locations were co-registered with the user's MRI using the nasion and left and right preauricular landmarks. A three-shell realistic-geometry head model with a conductivity ratio of 1:1/20:1 was generated using the boundary element method (BEM) implemented in the OpenMEEG toolbox.

The inverse operator for each session was generated according to the following theory. Eq. 5 depicts the linear system relating scalp and cortical activity, where ϕ(t) represents the scalp recorded EEG at time t, L the user- and session-specific leadfield, and J(t) the cortical current density at time t.

ϕ(t)=LJ(t) Eq. 5

Linear programming techniques can help stabilize the often ill-conditioned nature of the leadfield to find optimal estimates of the source distribution. In the current work we utilized Tikhonov regularization, (Eq. 6). This optimization suggests a solution J(t) that depends on various known parameters that include the sensor covariance matrix C, source covariance matrix R, regularization parameter), leadfield, and scalp EEG.

$${min}_{I} {‖ {C}^{- 1 / 2} ( φ ( t ) - LJ ( t ) ) ‖}_{2}^{2} + {λ}^{2} {‖ {R}^{- 1 / 2} J ( t ) ‖}_{2}^{2} , where \\ {λ}^{2} = \frac{tr ( {LRL}^{T} )}{tr ( C ) {SNR}^{2}} \qquad Eq . 6$$

The closed-form solution to Eq. 6, solving for an optimal source distribution is shown in Eq. 7 in the time domain and belongs to the family of minimum-norm estimates. Here, 20 seconds of resting-state EEG collected at the beginning of each session was used to compute a diagonal sensor covariance matrix C. The source covariance matrix was also a diagonal matrix with non-zero elements containing a depth-weighted reciprocal of source location power. This modification to the source covariance matrix forms the weighted minimum-norm estimate (WMNE).

Ĵ(t)=RL^{T}(LRL^{T}+λ²C)⁻¹ϕ(t) Eq. 7

This solution can be applied in the frequency domain by solving for both the real and imaginary frequency-specific cortical activity independently (45), and subsequently taking the magnitude at each cortical location (Eq. 8).

Ĵ_{Re}(f)=RL^{T}(LRL^{T}+λ²C_{Re})⁻¹ϕ_{Re}(f)

Ĵ_{IM}(f)=RL^{T}(LRL^{T}+λ²C_{IM})⁻¹ϕ_{IM}(f) Eq. 8

To utilize the spatial filtering properties of inverse imaging and extract task-related activity, the reconstructed cortical activity was subjected to both anatomical and functional constraints. The anatomical constraint is represented by limiting cortical activity to the central sensorimotor ROI previously described. The functional constraint is based on the data driven parcellation of the ROI into discretized, functionally coherent cortical clusters. Parcellation is particularly attractive for real-time applications as it improves the condition of the EEG inverse problem and reduces computation time (48). Parcellation was performed using the multivariate source prelocalization (MSP) algorithm using the MI without feedback data collected at the beginning of each session (48). Solving for the activity in each of these cortical clusters extends Eq. 8 to Eq. 9 where the subscript k represents the number of cortical parcels.

$${\hat{I}}_{k , Re} ( f ) = {R}_{k} {{L}_{k}^{T} ( ( {\sum }_{k} {L}_{k} {R}_{k} {L}_{k}^{T} ) + {λ}_{Re}^{2} {C}_{Re} )}^{- 1} {φ}_{Re} ( f ) \\ {\hat{I}}_{k , Im} ( f ) = {R}_{k} {{L}_{k}^{T} ( ( {\sum }_{k} {L}_{k} {R}_{k} {L}_{k}^{T} ) + {λ}_{Im}^{2} {C}_{Im} )}^{- 1} {φ}_{Im} ( f ) \qquad Eq . 9$$

Channel-Frequency Optimization

Each of the MI without feedback runs was analyzed individually to identify features used to control cursor movement in the two dimensions. For the sensor domain, the alpha power (8-13 Hz) at each electrode was extracted at a 1 Hz resolution using a Morlet wavelet technique. A stepwise linear regression was utilized with a forward inclusion step (p<0.01) and backward removal step (p<0.01) to find the electrodes and weights that best separate the two tasks used for each control dimension. This procedure was applied to frequency-specific R2 montages in the order of descending maximum values until at least one electrode survived the statistic thresholding. The weight of each selected electrode was set to −1 or +1 based on the sign of the regression beta coefficient. A weight of 0 was applied to all other electrodes not selected. If no electrodes were selected for any frequency, a default setup assigned −1 and +1 to the C3 and C4 electrodes, respectively for horizontal control, and −1 and −1 to both electrodes for vertical control.

For feature selection in the source domain, the MI EEG was first mapped to the cortical model according to Eq. 9. The stepwise linear regression procedure was applied to all ROI parcels and weights were assigned accordingly. If no parcels were selected, the default source setup was defined by assigning a weight of −1 to those parcels containing the left motor cortex hand knob and +1 to those containing the right motor cortex hand knob for horizontal control, and a weight of −1 to bilateral hand knob parcels for vertical control. These parcels were identified based on seed points assigned to the hand knobs (similar to (44)) by the operators prior to the experimental session.

Feature spread was calculated as the average Euclidian distance between the feature location and the lateral hand knob (source space) or C3/C4 electrode (sensor space). The hand knob location was defined as the average location of the previously mentioned seed points. The distance was also weighted by the magnitude of the feature weight to account for its strength. Distances were calculated for the left and right sides of the head individually and pooled together for each dimension.

Online Signal Processing

All online processing was performed using custom MATLAB (The Mathworks, Inc., MA, USA) scripts that communicated with BCI2000 using the FieldTrip buffer signal processing module. 57 electrodes covering the motor-parietal region of the scalp were utilized for online processing. The EEG was downsampled to 256 Hz and bandpass filtered between 8 and 13 Hz using a fourth-order Butterworth filter prior to common average referencing. The most recent 250 ms of data were analyzed and used to update the cursor velocity every 100 ms. The instantaneous control signal was computed as the weighted sum of the alpha power in the selected electrodes. If ϕF(f) represents the magnitude of the alpha power across the entire EEG montage at time window t, and x″ and x− are vectors containing the electrode weights (1s, −1s, and 0s) assigned during the optimization process, the instantaneous control signal for each dimension can be represented as:

C_{h,t}=xₕ^{T}ϕₜ(f) C_{v,t}=xᵥ^{T}ϕₜ(f) Eq. 10

The velocity of the cursor in each dimension was then derived by normalizing these values to zero mean and unit variance based on the values stored from the previous 30 seconds of online control in the respective dimension:

$${\vec{V}}_{h , t} = \frac{{C}_{h , t} - \overline{C} \text{?}}{σ \text{?}} {\vec{V}}_{v , t} = \frac{{C}_{v , t} - \overline{C} \text{?}}{σ \text{?}} \\ \text{?} \text{indicates text missing or illegible when filed} \qquad Eq . 11$$

The same procedure was performed for source control using the reconstructed cortical frequency information JhF (f) and the corresponding cortical cluster weights. Robotic arm positions were controlled via a custom C++ script which read and translated cursor positions into optimal joint angles.

Offline Data Analysis

CP data files contained cursor and target positions. These values were normalized to the screen size and used to obtain an error, defined as the Euclidean distance between the cursor and target, at each time point. The tracking correlation was computed as the Pearson correlation coefficient (ρ) between the target and cursor position time series. The mean squared error value was computed as the average of the error time series between these same two position vectors. The choice to use ρ2 (squared tracking correlation) was based on the concept of user control; signed values of ρ much less than 0 are superior to small positive values (e.g. −1 vs +0.01) as they suggest high quality control that is inverted, and that the simple inversion of weights can lead to high tracking performance. Furthermore, very few tracking correlation values were negative for both the original and physically constrained CP task. DT data files contained target and result codes for each trial used to compute percent valid correct values. Artifactual trials for both DT and CP runs were identified during online BCI control or by offline visual inspection of the EEG and removed from subsequent analysis.

MI without feedback data files contained the 128 channel EEG and MI task labels. Non-stationary high variance signals were initially removed from the raw EEG using the artifact subspace reconstruction (ASR) EEGlab plugin. Bad channels were spherically interpolated. The clean EEG was downsampled to 128 Hz, filtered between 5 and 30 Hz using a 4th order Butterworth filter, and re-referenced to the common average. The alpha (8-13 Hz) power was extracted from each channel using a Morlet wavelet for the time periods of 0.5-4.0 seconds after each stimulus presentation; a 0.5 second delay was included to account for user reaction time (after the visual cue). The alpha power in each channel and each frequency was regressed against the task labels. For the source domain, cortical alpha power was computed according to the Frequency-Domain Electrical Source Imaging section and regressed against the task labels.

Eye activity was extracted using independent component analysis (ICA). Clean EEG data for all tasks in the baseline and evaluation sessions were concatenated into separate data sets and decomposed using the extended infomax algorithm. The dimensionality of the data was first reduced using principle component analysis (PCA). The vertical and horizontal eye activity components (for Fig. S4 analysis) were identified as those containing high delta (1-4 Hz) activity and strong monopolar and bipolar frontal electrode projections, respectively (49). Not all sessions contained both distinct components meeting these criteria. Blink activity was computed as the variance of the (vertical/blink) independent component (IC) activation sequence during DT and CP control separately. To determine the influence of eye activity on BCI performance a regression analysis was performed between the vertical or horizontal eye activity IC activation sequence and target location in the corresponding dimension.

Statistical Analysis

Statistical analysis was performed using custom R and Matlab scripts. Effect sizes are reported throughout the manuscript as the point biserial correlation, |r| to highlight within group (e.g. training) and across condition (e.g. source vs. sensor, robotic arm vs. virtual cursor) differences. The point biserial correlation was computed according to Eq. 10, where MT and M % are the means of the two distributions being compared and SDpooled is the pooled standard deviation (d is also known as cohen's d).

$$r = \frac{d}{\sqrt{{d}^{2} + 4}} , d = \frac{{M}_{1} - {M}_{2}}{{SD}_{pooled}} \qquad Eq . 10$$

Unless otherwise stated, two-way repeated measures i were utilized with main effects of time and training task (DT vs CP), decoding domain (source vs. sensor), or control method (robotic arm vs. virtual cursor). All behavioral and electrophysiological metrics were first evaluated with the Shapiro-Wilk test to test for the normality of the residuals of a standard ANOVA. If the p-value of the majority of the all multiple comparisons was less than 0.05, a rank-transformed ANOVA was used. Otherwise, a standard ANOVA was used. If less than 10 multiple comparisons were made, a Tukey's HSD test was used to correct for multiple comparisons, and if greater than 10 comparisons, false discovery rate correction (p<0.05) was employed. A Mann-Whitney U test with Bonferroni correction for multiple comparisons was used for specific cases: comparing squared tracking correlation values (ρ2) of neural control with noise in the constrained CP task and comparing the feature spread in the source and sensor domains in naïve and experienced users.

Supplementary Materials

FIG. 7 displays example trials of neural virtual cursor tracking trajectories for the original continuous pursuit task. FIG. 7c illustrates the trajectory unwrapping method. First, the target positions were subtracted from the cursor positions (both between 0 and 1) to obtain an error time series. A −1 was added to cursor position indices when the error was greater than 0.5, and a +1 was added to those when the error was less than −0.5. These cases represent instances where the cursor deviated slightly from the target near an edge and wrapped to the other side of the workspace (red circles). Such behavior and dramatic changes in relative position can significantly penalize the correlation calculation, even though tracking performance is still quite good. The unwrapped trajectory therefore corrected for these cases by reconstructing accurate relative trajectories (red arrows).

FIG. 7A) Normalized cursor and target trajectories for 1D horizontal (left) and 1D vertical (right) trials. (B) Cursor and target trajectories for 2D trials. Red circles in the bottom row highlight instances of horizontal (left) and vertical (right) cursor edge wraps. (C) Unwrapped 2D trajectories for the trial in the bottom row of (B). Red arrows highlight where the unwrapping procedure mitigates tracking biases resulting from the edge wrapping procedure.

FIG. 8 Squared Tracking Correlation Histograms. (A) Histograms of squared tracking correlation values (ρ2) for the X-coordinate (left) and Y-coordinate (right) during 1D horizontal and 1D vertical trials, respectively. (B) Histograms of squared tracking correlation values (ρ2) for the X-coordinate (left) and Y-coordinate (right) during 2D trials. Histograms are composed of −350 trials each.

FIG. 9. Continuous Pursuit vs. Discrete Trial BCI Learning. (A) 1-dimensional (1D) horizontal and vertical performance values for the DT task at baseline and evaluation for the CP and DT training groups. (B) 1D horizontal and vertical performance values for the CP task at baseline and evaluation for the CP and DT training groups. The red dotted line indicates chance level. Bars indicate mean+standard error of the mean (SEM). The effect size, |r| is indicated under each pair of bars. Statistical analysis using a repeated measures two-way ANOVA (n=11 per group) with main effects of time (#p<0.05, ###p<0.005) and training task. Tukey's HSD post hoc test: * p<0.05, *** p<0.005.

FIG. 10 displays the group-level spatial and spectral characteristics of the vertical (a) and horizontal (b) eye movement EEG independent components (ICs). The timeseries of these ICs were utilized to determine if the user's gaze played a role in driving cursor movement (FIG. 2g). While eye activity in general was loosely correlated with cursor movement for both vertical and horizontal dimensions, (R2<0.1), it was significantly lower during the CP task compared to the DT task. FIG. 10A-10B: Regression output between the vertical (A) and horizontal (B) eye activity EEG independent component activation timeseries and target position. The EEG topography and power spectrum of the corresponding IC are displayed to the right. Bars indicate mean+SEM. Statistical analysis using a repeated measures two-way ANOVA (n=11 per group) with main effects of time and task (###p<0.005).

FIG. 11 Source vs. Sensor BCI Learning. (A) 1-dimensional (1D) horizontal and vertical performance values for the DT task at baseline and evaluation for the CP (sensor) and sCP (source) training groups. (B) 1D horizontal and vertical performance values for the CP task at baseline and evaluation for the CP (sensor) and sCP (source) training groups. The red dotted line indicates chance level. Bars indicate mean+SEM. The effect size, |r| is indicated under each pair of bars. Statistical analysis using a repeated measures two-way ANOVA (n=11 per group) with main effects of time (#p<0.05, ##p<0.01, ###p<0.005) and training neurofeedback domain. Tukey's HSD post hoc test: * p<0.05, *** p<0.005.

FIG. 12 highlights the procedure for deriving the spatial extent threshold for statistical testing from the squared error histograms. Gamma functions were fit to the histograms and the effect size at each bin was calculated. The extent at which the effect size changed from positive to negative was used as the spatial threshold.

FIG. 12. 2D CP Source vs. Sensor Spatial Threshold. A-C: Experienced user data (n=16). (A) Group-level squared-error histograms for 2D CP sensor and source cursor control (taken from FIG. 5b, d). (B) Group-level histograms fit with a gamma function. Goodness-of-fit values (GoF) are displayed in the inlay to the right. (C) Effect sizes between the source and sensor fitted histogram at each bin. The point at which the effect size change from positive to negative was defined as the extent threshold used for statistical testing. D-F: Naïve user data (n=13), same as A-C.

FIG. 13 Online 1D Horizontal CP Source vs. Sensor BCI Performance. A-C: Experienced user data (n=16). (A) Group-level squared-error histograms for 1D horizontal CP sensor and source cursor control. (B) Group-level histograms fit with a gamma function. Goodness-of-fit values (GoF) are displayed in the inlay to the right. (C) Effect sizes between the source and sensor fitted histograms at each bin. D-F: Naïve user data (n=13), same as A-C. (G) Scale drawing of the continuous paradigm workspace displaying the spatial threshold derived from for experienced (yellow) and naive (green) users derived from the fitted histogram effect size plots in C and F. FIG. 13H: Cursor dwell time within the spatial threshold for experienced (left) and naïve (right) users using the raw (top) and fitted (bottom) histogram data. Bars and circles indicate mean±SEM. Statistical analysis using a one- (naïve) or two-way (experienced) ANOVA with main effects of time, and time and decoding domain, respectively.

FIG. 14 Online 1D Vertical CP Source vs. Sensor BCI Performance. A-C: Experienced user data (n=16). (A) Group-level squared-error histograms for 1D vertical CP sensor and source cursor control. (B) Group-level histograms fit with a gamma function. Goodness-of-fit values (GoF) are displayed in the inlay to the right. (C) Effect sizes between the source and sensor fitted histograms at each bin. D-F: Naïve user data (n=13), same as A-C. (G) Scale drawing of the continuous paradigm workspace displaying the spatial threshold derived from for experienced (yellow) and naive (green) users derived from the fitted histogram effect size plots in C and F.

(H) Cursor dwell time within the spatial threshold for experienced (left) and naïve (right) users using the raw (top) and fitted (bottom) histogram data. Bars and circles indicate mean±SEM. Statistical analysis using a one- (naïve) or two-way (experienced) ANOVA with main effects of time, and time and decoding domain, respectively.

FIG. 15 Offline Source vs. Sensor Sensorimotor Modulation. (A) Conceptual illustration of the bilateral sensors (C3/C4) and cortical patches (left/right hand knobs) that are thought to best produce/capture various hand motor imagery task signals. B-C: Maximum R2 values found in the sensor and source sensorimotor locations identified in (A) for horizontal (B) and vertical (C) commands. Bars indicate mean+SEM. Statistical analysis using a rank-transformed one-way ANOVA with a main effect of decoding domain (n=13 naïve users) and a rank-transformed repeated measures two-way ANOVA with main effects of time and decoding domain (n=16 experienced users). Main effect of decoding domain: ##p<0.01, ###p<0.005.

What is claimed is:

1. A method of controlling an external device through a brain-computer interface comprising:
non-invasively obtaining a plurality of signals originating in the brain of a user while the user performs a task;
analyzing the plurality of signals;
extracting a control signal from the analyzed plurality of signals;
controlling the external device using the control signal.

2. The method of claim 1, wherein the plurality of signals is obtained via electroencephalography.

3. The method of claim 1, wherein the plurality of signals is obtained via magnetoencephalography.

4. The method of claim 1, wherein the plurality of signals is selected from the group consisting of electrical, magnetic, or hemodynamic signals.

5. The method of claim 1, wherein the external device is selected from a group consisting of a computer, robotic device, a neuroprosthetic limb, a wheelchair, a drone, a smartphone, or an assistive device.

6. The method of claim 1, further comprising:
estimating the neural sources generating the plurality of signals through real-time source imaging.

7. The method of claim 1, wherein non-invasively obtaining the plurality of signals comprises:
using non-invasive neuroimaging.

8. The method of claim 7, wherein the non-invasive neuroimaging comprises real-time electrical source imaging.

9. The method of claim 7, wherein using non-invasive neuroimaging comprises:
isolating and evaluating sensor and source signals during online processing.

10. The method of claim 1, wherein analyzing the plurality of signals comprises:
processing the plurality of signals in the temporal, spatial, and spectral domains.

11. The method of claim 1, wherein analyzing the plurality of signals comprises:
decoding the user's mental intent or state based on the spatio-temporal-spectral signatures contained within the plurality of signals.

12. The method of claim 11, wherein the plurality of signals is processed to identify brain signals representing a user's motor or mental intention.

13. The method of claim 11, further comprising:
extracting spatio-temporal-spectral features from the plurality of signals; and
identifying the control signal using linear or non-linear classifiers.

14. The method of claim 13, wherein the linear classifier can include at least one of simple linear combination of powers, linear discriminative analysis, and support vector machine with linear kernels.

15. The method of claim 13, wherein the non-linear classifier can include at least one of neural networks, deep learning networks, and support vector machine with nonlinear kernels.

16. A method of training a user to control an external device through a brain-computer interface comprising:
directing the user to engage in a continuous pursuit task wherein the user performs motor imagination to chase a randomly moving target;
non-invasively obtaining a plurality of signals originating in the brain while the user engages in the continuous pursuit task; and
analyzing the plurality of signals.

17. The method of claim 16, wherein the moving target comprises at least one of a virtual object appearing on a screen and a real object appearing in physical space.

18. The method of claim 16, further comprising:
identifying relevant spatio-temporal-spectral features from the plurality of signals.

19. The method of claim 16, further comprising:
producing a continuous estimate of motor or mental intention.

20. The method of claim 1, further comprising:
estimating a motor state or mental state using continuous pursuit signals, wherein estimating is online and adaptive.

21. The method of claim 16, further comprising:
estimating a motor state or mental state using continuous pursuit signals, wherein estimating is online and adaptive.
